(19) United States
(12) Patent Application Publication
(10) Pub. No.: US 2021/0065844 A1
(43) Pub. Date: Mar. 4, 2021

(54) METHODS TO DETERMINE THE SENSITIVITY PROFILE OF A BACTERIAL STRAIN TO A THERAPEUTIC COMPOSITION

(71) Applicant: Adaptive Phage Therapeutics, Inc., Gaithersburg, MD (US)

(72) Inventor: Carl Merril, Bethesda, MD (US)

(73) Assignee: Adaptive Phage Therapeutics, Inc., Gaithersburg, MD (US)

(21) Appl. No.: 16/644,203

(22) PCT Filed: Sep. 5, 2018
(86) PCT No.: PCT/US2018/049481

§ 371 Date: Mar. 4, 2020

Related U.S. Application Data
(60) Provisional application No. 62/673,162, filed on May 18, 2018.
(60) Provisional application No. 62/597,151, filed on Dec. 11, 2017.
(60) Provisional application No. 62/554,529, filed on Sep. 5, 2017.

(51) Int. Cl. G16B 20/00; G16B 40/00; G16B 40/30; G16B 40/20; G06N 20/00

(52) U.S. Cl. CPC G16B 20/00; G16B 40/00; G06N 20/00; G16B 40/20; G16B 40/30

(57) ABSTRACT

Methods and systems for pattern search and analysis to identify and select therapeutic molecules that can be used to treat bacterial infections or contaminations. Examples include methods and systems for pattern search and analysis to identify and select bacteriophage based on comparison of the genomes of a query bacterium and/or a query phage strain to a therapeutic molecule-host training set of bacterial strains and/or phage strains in which the phage strains (or other therapeutic molecules) have been shown to have the capacity to act as an antibacterial agent by either killing, replicating in, lysing and/or inhibiting the growth of the bacterial strains in the training set. Therapeutic compositions, including phage, identified using the methods described herein can then be used to treat bacterial infections in a subject and/or contamination in the environment.

### BACKGROUND OF THE INVENTION

### Field of the Invention

The invention relates to cell-free methods and kits useful for predicting a bacterium's sensitivity to a therapeutic composition, including a phage, an antibiotic, and/or other bactericidal compound. Synergist bactericidal activity between therapeutic compositions can also be predicted using the cell-free methods and kits described herein.

### Discussion of the Related Art

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Multiple drug resistant (MDR) bacteria are emerging at an alarming rate. Currently, it is estimated that at least 2 million infections are caused by MDR organisms every year in the United States leading to approximately 23,000 deaths. Moreover, it is believed that genetic engineering and synthetic biology may also lead to the generation of additional highly virulent microorganisms.

For example, Staphylococcus aureus are gram positive bacteria that can cause skin and soft tissue infections (SSTI), pneumonia, necrotizing fasciitis, and blood stream infections. Methicillin resistant S. aureus (“MRSA”) is an MDR organism of great concern in the clinical setting as MRSA is responsible for over 80,000 invasive infections, close to 12,000 related deaths, and is the primary cause of hospital acquired infections. Additionally, the World Health Organization (WHO) has identified MRSA as organisms of international concern.

In view of the potential threat of rapidly occurring and spreading virulent microorganisms and antimicrobial resistance, alternative clinical treatments against bacterial infection are being developed. One such potential treatment for MDR infections involves the use of phage. Bacteriophages (“phages”) are a diverse set of viruses that replicate within and can kill specific bacterial hosts. The possibility of harnessing phages as an antibacterial was investigated following their initial isolation early in the loth century, and they have been used clinically as antibacterial agents in some countries with some success. Notwithstanding, phage therapy was largely abandoned in the U.S. after the discovery of penicillin, and only recently has interest in phage therapeutics been renewed.

The successful therapeutic use of phage depends on the ability to administer a phage strain that can kill or inhibit the growth of a bacterial isolate associated with an infection. In addition, given the mutation rate of bacteria and the narrow host range associated with phage strains, a phage strain that is initially effective as an antibacterial agent can quickly become ineffective during clinical treatment as the initial target bacterial host either mutates or is eliminated and is naturally replaced by one or more emergent bacterial strains that are resistant to the initial phage employed as an antibacterial agent.

Empirical laboratory techniques have been developed to screen for phage susceptibility on bacterial strains. However, these techniques are time consuming and are dependent upon obtaining a bacterial growth curve for each specific strain of bacterium. For example, phage stains are currently screened for their capacity to lyse (kill) or inhibit bacterial growth by testing individual phage strains against a specific patient's bacterial isolate using either liquid cultures or bacterial lawns grown on agar media. This growth requirement cannot be quickened and susceptibility results are generated only after hours, and in some cases, days of screening. This delay in obtaining susceptibility results can lead to delay of treatment and complications for a patient suffering from a systemic bacterial infection.

Thus, there is a need to develop rapid screening methods for predicting bacterial susceptibility to specific phage stains that do not rely on the growth of bacterial cultures.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The invention relates to cell-free based kits and methods for rapidly predicting the sensitivity of a bacterium to a therapeutic composition, such as one or more phage strains, one or more antibiotic, and/or one or more other bactericidal compound, or any combination thereof. For example, the invention confers improvements in both processing speed and the capacity of a phage strain to successfully infect a specific bacterial isolate, thereby eliminating reliance on bacteria growth curves. The generation of a trained machine learning therapeutic composition model, including one or more phage models, bacterial host models, antibiotic models, and/or other bactericidal compound models enable rapid generation of clinically predictive bacterial sensitivity results to specific phage, antibiotic(s), and/or therapeutic treatment or any combination thereof.

Preferred bacterial strains that can be used to generate the machine learning model(s) include but are not limited to, the ESKAPE pathogens such as strains of salmellonella, Enterococcus faecium, Staphylococcus aureus, Klebsiella pneumonia, Acinetobacter baumannii, Pseudomonas aeruginosa, and Enterobacter sp. The methods and kits of the invention are based on the discovery that by using machine learning, genomic patterns can be identified in specific bacteria, and in some embodiments, in specific phage that that are predictive for that bacteria's susceptibility to be either killed or inhibited by a specific phage, an antibiotic, and/or other bactericidal compounds, including combinations of these therapeutic compositions. These genomic sequence patterns which correlate with a sensitive vs. resistant phenotypes can be used to predict whether a subsequently tested query bacterium will also be sensitive or resistant to therapeutic compositions, including, but not limited to a particular phage strain, an antibiotic, and/or other bactericidal compound and/or any combination thereof. In preferred embodiments, these “predictive genomic patterns” in either a bacteria's genome and/or in combination with a phage's genome can function as a diagnostic tool, predicting a bacteria's sensitivity and/or resistance to phage strains. Moreover, these predictive genomic patterns can also be used to identify synergistic combinations between therapeutic compositions, and preferably between phage strains, antibiotics and/or other bactericidal compounds. In one embodiment, by applying machine learning and pattern recognition to phage-bacterial training set of different bacterial strains in combination with sets of phage stains, a query bacterial genome can be compared to the phage-host training sets and predicted sensitivity to phage strains can be made without requiring cell culture growth. This similar approach can also be used for any therapeutic composition (such as an antibiotic or other bactericidal compound) to predict sensitivity of the bacterial strain to the therapeutic composition (including combinations thereof) without requiring cell culture growth.

Broadly, the genomes of a plurality (for example hundred or multiple hundreds) of different bacterial strains along with experimentally derived bacterial host sensitivity profiles to the plurality of therapeutic composition are sequenced and the generated sequence data is analyzed and compared using computer-implemented machine learning and/or pattern recognition software known in the art to classify and identify patterns of identity between the bacterial genomes. These patterns of identity are then correlated with sensitive vs. resistant vs. synergistic therapeutic composition host phenotypes. Preferably, programs that employ artificial intelligence, including programs that employ tools such as Bayesian machine learning and/or Neural networks (e.g., searching for patterns within the genomes) can be used to classify regions of identity and/or high similarity which correlate with sensitivity/resistant/synergistic therapeutic composition host profiles. Both supervised and unsupervised learning methods can be used.

In one example, the genomes of a plurality (for example hundred or multiple hundreds) of different bacterial strains, and in preferred embodiments in combination with genomes of phage strains along with experimentally derived bacterial phage-host sensitivity profiles to the plurality of phage strains are sequenced and the generated sequence data is analyzed and compared using computer-implemented machine learning and/or pattern recognition software known in the art to classify and identify patterns of identity between the bacterial genomes and between the phage genomes. These patterns of identity are then correlated with sensitive vs. resistant vs. synergistic phage-host phenotypes. Preferably, programs that employ artificial intelligence, including programs that employ tools such as Bayesian machine learning and/or Neural networks (e.g., searching for patterns within the genomes) can be used to classify regions of identity and/or high similarity which correlate with sensitivity/resistant/synergistic host-phage profiles. These models can be combined with host models generated for other therapeutic compositions such as antibiotics and/or other bactericidal compounds, to identify those combinations that would have the most effective therapeutic potential. Both supervised and unsupervised learning methods can be used.

For example, in identifying genomic patterns in common between the bacterial strains, Block 130 shown in FIG. 1A uses computational methods to train a machine learning model (e.g., statistical methods, supervised learning, reinforcement learning, unsupervised learning, feature detection, artificial intelligence methods, neural network models, bioinformatics methods, etc.). In some embodiments the model is trained to recognize common and dissimilar patterns in genomic sequences between bacterial strains and/or between phage strains, or between bacterial stains with sensitivity to a therapeutic composition. These patterns are then characterized with the phage-host sensitivity data to label these similar and dissimilar sequences, as shown in Blocks 150 and 160, to generate the phage-host machine learning model. In some embodiments the phage-host sensitivity sequences may also be saved (Block 180).

The computational methods for identifying genomic patterns, characterizing therapeutic composition sensitivity data (eg phage-host sensitivity data, antibiotic-host sensitivity data, bactericide-host sensitivity data and/or sensitivity data of combinations) and/or selecting a sensitive therapeutic composition including a phage, an antibiotic, a bactericide, and combinations(as illustrated in Blocks 130, 140, 150, 160, 170, 180) can additionally or alternatively utilize any other suitable algorithms in performing these steps. For example, the algorithm(s) can be characterized by a learning style including any one or more of: supervised learning (e.g., using logistic regression, using back propagation neural networks), unsupervised learning (e.g., using an Apriori algorithm, using K-means clustering), semi-supervised learning, reinforcement learning (e.g., using a Q-learning algorithm, using temporal difference learning), and any other suitable learning style. In some embodiments supervised learning methods use the sequences as inputs and the therapeutic sensitivity data (eg phage-host sensitivity data, antibiotic-host sensitivity data, bactericide-host sensitivity data and/or sensitivity data of combinations) as the output data (target). In some embodiments semi-supervised learning methods may comprise unsupervised learning of sequences (e.g. clustering) followed by feature detection using the phage-host sensitivity data. The sequence data may be sequence data of a plurality of bacterial strains, or both sequence data of a plurality of bacterial strains and a plurality of phage strains. Furthermore, the algorithm(s) can implement any one or more of: a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4·5, chi-squared automatic interaction detection, decision stump, random forest, multivariate adaptive regression splines, gradient boosting machines, etc.), a Bayesian method (e.g., naive Bayes, averaged one-dependence estimators, Bayesian belief network, etc.), a kernel method (e.g., a support vector machine, a radial basis function, a linear discriminant analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolutional network method, a stacked auto-encoder method, etc.), a dimensionality reduction method (e.g., principal component analysis, partial least squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and any suitable form of algorithm. In some embodiments the machine learning method is trained to identify one or more therapeutic compositions including phages, antibiotics, bactericides, other therapeutic molecules, or combinations that are estimated to either kill or inhibited the bacteria present in a sample without explicitly identifying specific genomic sequences, or at least, without explicitly outputting a specific genomic sequence. That is whilst such machine learning methods and classifiers may be trained on and utilise sequence data, the specific sequences that lead to a classification decision may not be readily apparent, and may be stored in an internal model or as an internal set of weights and/or parameters that the method uses to classify an input sequence. In some embodiments the machine learning method receives sequence data from a target bacteria as input and outputs one or more therapeutic compositions and an estimate of the specificity of each therapeutic composition against the target bacteria (therapeutic specificity). This may include phages and an estimate of the specificity of each phage against the target bacteria (phage-host specificity), antibiotics and an estimate of the specificity of each antibiotic against the target bacteria (antibiotic-host specificity), bactericides and an estimate of the specificity of each bactericide against the target bacteria (bactericide-host specificity), or combinations and an estimate of the specificity of the combination against the target bacteria. In some embodiments the machine learning model is a deep learning system that classifies a sample using multiple internal layered classifiers and/or neural nets trained on suitable training data, without explicitly identifying specific genomic sequences. Deep learning classifiers typically require large amounts of training data. Thus in some embodiments a deep learning classifier is developed or refined over time as additional clinical samples and outcomes are received. In some embodiments the machine learning methods may produce probabilistic estimates of the effectiveness or specificity of a phage against an input bacteria sequence.

Once the Therapeutic Composition machine learning model has been generated, a query bacterium can be processed to predict therapeutic specificity (eg phage-host specificity, Antibiotic Specificity, Bactericide Specificity and/or specificity of combinations), all without the need for wet-laboratory data. Therapeutic compositions, such as a phage, an Antibiotic, a Bactericide, a therapeutic molecule or a combination identified as specific to the query bacterium can then be used as a therapeutic or decontaminant. In further preferred embodiments, multiple therapeutic compositions (eg one or more phage strains, one or more antibiotics, one or more bactericides, and/or one or more therapeutic molecules) can be identified in the methods described herein and used to generate a cocktail that can then be used to treat a bacterial infection or contamination. In preferred embodiments, the multiple therapeutic compositions (eg multiple phage strains, or various combinations of phage, antibiotics, bactericides and/or therapeutic molecules) in the cocktail have different patterns of specificity—which may help in reducing the incidence of bacterial phage resistance.

In a further preferred embodiment, the patterns of similarity and/or identity (also referred to collectively as “predictive patterns” or Therapeutic Composition Sensitivity Sequences including “phage host sensitivity sequences”, “Antibiotic-Host Sensitivity Sequences”, and “Bactericide-Host Sensitivity Sequences”) are used to classify the bacteria strains into at least 2, at least 3, at least 4 major therapeutic-host sensitivity profile groups, such as phage-host sensitivity profile groups, antibiotic-host sensitivity profile groups, other bactericidal compound-host sensitivity profile groups, and/or synergistic therapeutic molecule-host sensitivity profile groups.

In further preferred embodiments, cocktails comprising a mixture of therapeutic compositions selected from some or all of the sensitivity groups have varying sensitivity profiles can be generated. These cocktails can be used to treat a bacterial infection or contamination. In preferred embodiments, therapeutic composition selection may enhance resistance to the development of bacterial resistance to that cocktail.

In preferred embodiments, the bacterial and/or phage genomes are sequenced using rapid sequencing techniques known to the skilled artisan. Examples of such techniques, include, but are not limited to rapid nanopore genomic sequencing.

Preferably, the method comprises an additional step of sub-typing strains identified as having a specific therapeutic-host sensitivity profile according to sensitivity. Thus, for example, a bacterial strain or strains identified as being sensitive, insensitive, or intermediate sensitivity to a phage, antibiotic, bactericide or combination, can be sub-typed and further classified according to phage, antibiotic, bactericide or combination sensitivity.

In one embodiment a computational method for generating a therapeutic composition machine learning model is described, wherein the method comprises:
(a) compiling data from a plurality of bacterial strains in a computer database system, wherein the data comprises genomic sequence data of a plurality of bacterial strains;
(b) training a machine learning model using at least the genomic sequence data of a plurality of bacterial strains on a CPU and a memory unit of a computer system; and
(c) storing a therapeutic composition machine learning model configured to receive a query bacterial genome and select at least one therapeutic composition estimated to be sensitive to the bacterial genome based on the trained machine learning model.

The at least one therapeutic composition estimated to be sensitive to the bacterial genome based on the trained machine learning model may comprise one or more phage, antibiotic, bactericide, therapeutic molecule or combination estimated to be sensitive to the bacterial genome based on the trained machine learning model

In one embodiment, the least one therapeutic composition comprises at least one phage
and in step (a) the data further comprises
   genomic sequence data of a plurality of phage strains;
and in step (b) training a machine learning model uses at least the genomic sequence data of a plurality of bacterial strains and the genomic sequence data of a plurality of phage strains on a CPU and a memory unit of a computer system; and
in step (c) the therapeutic composition machine learning model configured to receive a query bacterial genome is configured to select at least one phage estimated to be sensitive to the bacterial genome based on the trained machine learning model.

In some embodiments, wherein the machine learning model generates therapeutic composition sensitivity sequences. These may be phage-host sensitivity sequences, antibiotic-host sensitivity sequences, bactericide-host sensitivity sequences or other therapeutic molecule-host sensitivity sequences. In some embodiments the method further comprises receiving experimentally derived therapeutic composition-host sensitivity profiles of the bacterial strains experimentally derived from a plurality of therapeutics, and generating the therapeutic composition sensitivity sequences comprises performing feature detection using the therapeutic composition-host sensitivity profiles comprising:
(1) identifying common genomic sequence patterns shared between the bacterial strains having similar or identical therapeutic composition-host sensitivity profiles; and/or
(2) identifying dissimilar genomic sequence patterns shared between the bacterial strains having dissimilar therapeutic composition-host sensitivity profiles;
and training the model further comprises characterizing each bacterial strain by associating the therapeutic composition Sensitivity Sequences with therapeutic composition-host sensitivity profiles and generating a prediction profile for therapeutic composition-host specificity for each bacterial strain.

In one embodiment the method further comprises receiving additional genomic sequence data and therapeutic composition-host sensitivity profiles for a plurality of bacteria and refining the machine learning model. In one embodiment the the machine learning model is trained in an unsupervised process.

In one embodiment, a computational method for generating a therapeutic composition machine learning model is described wherein the method comprises:
(a) compiling data from a plurality of bacterial strains in a computer database system, wherein the data comprises (1) genomic sequence data of a plurality of bacterial strains; and (2) experimentally derived therapeutic composition-host sensitivity profiles of the bacterial strains experimentally derived from a plurality of therapeutic;
(b) training a machine learning model using the genomic sequence data of a plurality of bacterial strains and the experimentally derived therapeutic composition-host sensitivity profiles on a CPU and a memory unit of a computer system;
(c) storing a therapeutic composition machine learning model configured to receive a query bacterial genome and select at least therapeutic composition comprising one or more phage, antibiotic, bactericide, therapeutic molecule or combination estimated to be sensitive to the bacterial genome based on the trained machine learning model.

The at least therapeutic composition may comprise at least one phage, at least on antibiotic, at least one bactericide or a combination. The therapeutic composition-host sensitivity profiles may be phage-host sensitivity profiles, antibiotic-host sensitivity profiles, bactericide-host sensitivity profiles or other therapeutic molecule-host sensitivity profiles. These may be experimentally derived a plurality of phage, antibiotics, bactericides, therapeutic molecules, etc.

In preferred embodiments, the bacterial and/or phage genomes are sequenced using rapid sequencing techniques known to the skilled artisan. Examples of such techniques, include, but are not limited to rapid nanopore genomic sequencing.

In preferred embodiments, the machine-learning and pattern recognition analysis incorporates Neural network analysis, including deep Neural Network learning or Artificial Neural network analysis, or classic models, such as, Bayesian, Gaussian analysis, regression analysis, and/or Tree analysis.

In further preferred embodiment, the experimentally derived therapeutic composition-host sensitivity data is generated by performing a plaque assay. In preferred embodiments, the size, cloudiness, clarity and/or presence of a halo of the plaque is measured. In other preferred embodiments, the experimentally derived therapeutic composition-host sensitivity data is generated using a photometric assay selected from the group consisting of fluorescence, absorption, and transmission assays.

In one embodiment the machine learning model is updated by receiving (1) additional genomic sequence data of a plurality of bacterial strains; and (2) experimentally derived therapeutic composition-host sensitivity profiles of the additional bacterial strains experimentally derived from a plurality of therapeutic compositions. The received information is used to retrain (or update) the machine learning model.

A computer implemented method for predicting therapeutic composition-host sensitivity of a query bacterium, the method comprising: (a) receiving the phage-host machine learning model described herein, (b) receiving genomic sequence data of the query bacterium; and (c) predicting a therapeutic composition-host sensitivity of the query bacterium based on the machine learning model after training. In some embodiments, the machine learning model is trained in an unsupervised process, supervised process and/or incorporates Neural network analysis, including deep Neural Network learning or Artificial Neural network analysis, or classic models, such as, Bayesian, Gaussian analysis, regression analysis, and/or Tree analysis.

In further preferred embodiments, therapeutic compositions are selected by a method comprising selecting at least one therapeutic composition based on a profile match score generated from a query bacterial genome provided as input to the therapeutic composition-host machine learning model, wherein a higher profile match score represents a higher therapeutic composition-host sensitivity. The machine learning and pattern recognition used in this method incorporates Neural network analysis, including deep Neural Network learning or Artificial Neural network analysis, or classic models, such as, Bayesian, Gaussian analysis, regression analysis, and/or Tree analysis.

Selection of multiple phage (and/or multiple other therapeutic compositions) are contemplated as well as formulation of selected phage (and other therapeutic compositions) in a pharmaceutically acceptable composition.

In preferred embodiments, the compositions of selected therapeutic compositions comprise therapeutic compositions having different host range, comprise a mixture of therapeutic compositions having broad host range and therapeutic compositions having a narrow host range, and/or act synergistically with one another.

The therapeutic compositions described herein can have a number of activities on bacteria, including but not limited to: (a) delay in bacterial growth; (b) lack of appearance of phage-resistant bacterial growth; (c) less virulent; (d) regain sensitivity to one or more drugs; and/or (e) display reduced fitness for growth in the subject.

Compositions comprising the therapeutic compositions described herein are preferred embodiments, as well as a method of treating a subject having a bacterial infection or an environmental contamination using the compositions as described herein. In preferred embodiments, the bacterial infection or bacterial contamination to be treated is selected from the group consisting of wound infections, post-surgical infections, and systemic bacteremias. In further preferred embodiments, the bacterial infection/contamination is selected from infection caused by an “ESKAPE” pathogens (Enterococcus faecium, Staphylococcus aureus, Klebsiella pneumonia, Acinetobacter baumannii, Pseudomonas aeruginosa, and Enterobacter sp).

In further embodiments, the system described herein comprises: one or more processors; memory; and one or more programs, wherein the one or more programs are stored in the memory and configured to be executed by the one or more processors, the one or more programs including instructions for carrying out any of the method described herein. In preferred embodiments, the bacterial strains of the plurality of bacterial strains, the query bacterial genome, and/or the bacterial infection of the methods or compositions as described herein is (are) selected from a) multidrug resistant bacteria; b) a clinical bacterial isolate causing infection in a subject; c) a clinical bacterial isolate causing infection in a subject and is multidrug resistant; d) obtained from bona-fide human infections; or e) obtained from a diverse source. The diverse source can be selected from, in preferred embodiments, the group consisting of soil, water treatment plants, raw sewage, sea water, lakes, rivers, streams, standing cesspools, animal and human intestines, and fecal matter.

Also as described, the therapeutic composition-host machine learning model created according to any of the methods described herein as well as use of such therapeutic composition-host machine learning model to predict therapeutic composition-host sensitivity to a query bacteria.

### BRIEF DESCRIPTION OF THE FIGURES

The objects and features of the invention can be better understood with reference to the following detailed description and accompanying drawings.

FIG. 1A provides a flow diagram of generating a therapeutic composition-host training set of a plurality of bacterial strains.

FIG. 1B provides a flow diagram illustrating the machine learning module will be trained.

FIG. 1C provides a flow diagram illustrating unsupervised machine learning module and updating of the model as additional data becomes available.

FIG. 1D provides a flow diagram illustrating iterative supervised machine learning involving a training set, validation set, and test set to generate a machine learning model.

FIG. 2A is a flow diagram of predicting a therapeutic composition-host specificity profile for a query bacterium using the therapeutic composition-host training set generated according to FIGS. 1A to 1D. FIG. 2A also shows the additional selection of a therapeutic composition step.

FIG. 3 illustrates an exemplary machine learning model useful for the methods and systems described herein.

FIG. 4A illustrates an exemplary architecture of deep learning model comprising multiple internal layers for use in the method and systems described herein.

FIG. 4B illustrates connections between neurons in layers in a deep learning model for use in the method and systems described herein.

Like reference numbers and designations in the various drawings indicate like elements.

### DETAILED DESCRIPTION

The following definitions are provided for specific terms which are used in the following written description.

### DEFINITIONS

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules. “A phage cocktail” can mean at least one phage cocktail, as well as a plurality of phage cocktails, i.e., more than one phage cocktail. As understood by one of skill in the art, the term “phage” can be used to refer to a single phage or more than one phage.

The present invention can “comprise” (open ended) or “consist essentially of” the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention.

As used herein, a “subject” is a vertebrate, preferably a mammal, more preferably a human. Mammals include, but are not limited to, murines, simians, humans, farm animals, sport animals, and pets. In other preferred embodiments, the “subject” is a rodent (e.g., a guinea pig, a hamster, a rat, a mouse), murine (e.g., a mouse), canine (e.g., a dog), feline (e.g., a cat), equine (e.g., a horse), a primate, simian (e.g., a monkey or ape), a monkey (e.g., marmoset, baboon), or an ape (e.g., gorilla, chimpanzee, orangutan, gibbon). In other embodiments, non-human mammals, especially mammals that are conventionally used as models for demonstrating therapeutic efficacy in humans (e.g., murine, primate, porcine, canine, or rabbit animals) may be employed. Preferably, a “subject” encompasses any organisms, e.g., any animal or human, that may be suffering from a bacterial infection, particularly an infection caused by a multiple drug resistant bacterium.

As understood herein, a “subject in need thereof” includes any human or animal suffering from a bacterial infection, including but not limited to a multiple drug resistant bacterial infection. Indeed, while it is contemplated herein that the methods of the instant invention may be used to target a specific pathogenic species, the method can also be used against essentially all human and/or animal bacterial pathogens, including but not limited to multiple drug resistant bacterial pathogens. Thus, in a particular embodiment, by employing the methods of the present invention, one of skill in the art can design and create personalized therapeutic compositions (for example phage and/or phage/antibiotic cocktails) against many different clinically relevant bacterial pathogens, including multiple drug resistant (MDR) bacterial pathogens.

As understood herein, an “effective amount” of a pharmaceutical composition refers to an amount of the composition suitable to elicit a therapeutically beneficial response in the subject, e.g., eradicating a bacterial pathogen in the subject. Such response may include e.g., preventing, ameliorating, treating, inhibiting, and/or reducing one of more pathological conditions associated with a bacterial infection.

The term “dose” or “dosage” as used herein refers to physically discrete units suitable for administration to a subject, each dosage containing a predetermined quantity of the active pharmaceutical ingredient calculated to produce a desired response.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5 fold, and more preferably within 2 fold, of a value. Unless otherwise stated, the term “about” means within an acceptable error range for the particular value, such as ±1-20%, preferably ±1-10% and more preferably ±1-5%. In even further embodiments, “about” should be understood to mean+/−5%.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

All ranges recited herein include the endpoints, including those that recite a range “between” two values. Terms such as “about,” “generally,” “substantially,” “approximately” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

Where used herein, the term “and/or” when used in a list of two or more items means that any one of the listed characteristics can be present, or any combination of two or more of the listed characteristics can be present. For example, if a composition is described as containing characteristics A, B, and/or C, the composition can contain A feature alone; B alone; C alone; A and B in combination; A and C in combination; B and C in combination; or A, B, and C in combination.

As used herein, a “therapeutic composition” is any molecule that can be used to infect, kill, or inhibit the growth of a bacterium. Examples of such therapeutic compositions, include, but are not limited to phage, antibiotics, bactericidal compounds, and other therapeutic molecules (such as small molecules or biologics) that have bactericidal activity.

The term “sensitive” or “sensitivity profile” means a bacterial strain that is sensitive to infection and/or killing and/or in growth inhibition by a therapeutic compositions. For example, the term “phage sensitive” or “phage sensitivity profile” means a bacterial strain that is sensitive to infection and/or killing by phage and/or in growth inhibition.

The term “insensitive” or “resistant” or “resistance” or “resistant profile” is understood to mean a bacterial strain that is insensitive, and preferably highly insensitive to infection and/or killing and/or growth inhibition by a therapeutic composition. For example, the term “phage insensitive” or “phage resistant” or “phage resistance” or “phage resistant profile” is understood to mean a bacterial strain that is insensitive, and preferably highly insensitive to infection and/or killing by phage and/or growth inhibition.

The term “intermediate sensitivity” is understood to mean a bacterial strain that exhibits a sensitivity to infection and/or killing and/or growth inhibition by a therapeutic composition that is in between the sensitivity of sensitive and insensitive strains to a therapeutic composition. For example, the term “intermediate phage sensitivity” is understood to mean a bacterial strain that exhibits a sensitivity to infection and/or killing and/or growth inhibition by a phage that is in between the sensitivity of phage sensitive and phage insensitive strains.

As used herein, “predictive patterns”, “therapeutic composition-host sensitivity sequences” or “phage-host sensitivity sequences” are genomic patterns identified in the plurality of bacterial strains and/or in the plurality of phage strains making up the training sets as correlating with a “sensitivity profile”, “resistant profile”, or “intermediate sensitivity profile” of a bacterium.

As used herein, a “therapeutic composition-host specificity profile” is used interchangeably with a “therapeutic composition-host sensitivity profile” and comprises data relating to a bacterium's sensitivity or resistance to a plurality of different therapeutic compositions. For example, a “phage-host specificity profile” is used interchangeably with a “phage-host sensitivity profile” and comprises data relating to a bacterium's sensitivity or resistance to a plurality of different phage. The therapeutic composition-host specificity profile can be experimentally derived (such as is used for the therapeutic composition-host training set) or predictive (see Block 220) from performing the method as described herein.

A “therapeutic composition cocktail”, “therapeutically effective composition cocktail”, or like terms as used herein are understood to refer to a composition comprising a plurality of therapeutic compositions such as composed of one or more phages, antibiotics, or bactericides, which can provide a clinically beneficial treatment for a bacterial infection when administered to a subject in need thereof. In some embodiments “therapeutic phage cocktail”, “therapeutically effective phage cocktail”, “phage cocktail” will refer to a composition comprising a plurality of phage. Preferably, therapeutically effective therapeutic composition cocktails are capable of infecting the infective parent bacterial strain as well as the emerging resistant bacterial strains that may grow out after elimination of the parent bacterial strain.

As used herein, the term “composition” encompasses “therapeutic composition cocktails”, such as for example, “phage cocktails”, “antibiotic cocktails” and/or “other bactericidal compound cocktails” (and combinations of phage, antibiotics, and bactericides) as disclosed herein which include, but are not limited to, pharmaceutical compositions comprising a plurality of therapeutic compositions, such as a plurality of purified phages. “Pharmaceutical compositions” are familiar to one of skill in the art and typically comprise active pharmaceutical ingredients formulated in combination with inactive ingredients selected from a variety of conventional pharmaceutically acceptable excipients, carriers, buffers, and/or diluents. The term “pharmaceutically acceptable” is used to refer to a non-toxic material that is compatible with a biological system such as a cell, cell culture, tissue, or organism. Examples of pharmaceutically acceptable excipients, carriers, buffers, and/or diluents are familiar to one of skill in the art and can be found, e.g., in Remington's Pharmaceutical Sciences (latest edition), Mack Publishing Company, Easton, Pa. For example, pharmaceutically acceptable excipients include, but are not limited to, wetting or emulsifying agents, pH buffering substances, binders, stabilizers, preservatives, bulking agents, adsorbents, disinfectants, detergents, sugar alcohols, gelling or viscosity enhancing additives, flavoring agents, and colors. Pharmaceutically acceptable carriers include macromolecules such as proteins, polysaccharides, polylactic acids, polyglycolic acids, polymeric amino acids, amino acid copolymers, trehalose, lipid aggregates (such as oil droplets or liposomes), and inactive virus particles. Pharmaceutically acceptable diluents include, but are not limited to, water, saline, and glycerol.

Bacteria to be treated using the cocktails and compositions described herein include any bacterial pathogen that poses a health threat to a subject. These bacterial include, but are not limited to the “ESKAPE” pathogens (Enterococcus faecium, Staphylococcus aureus, Klebsiella pneumonia, Acinetobacter baumannii, Pseudomonas aeruginosa, and Enterobacter sp), which are often nosocomial in nature and can cause severe local and systemic infections. Among the ESKAPE pathogens, A. baumannii is a Gram-negative, capsulated, opportunistic pathogen that is easily spread in hospital intensive care units. Many A. baumannii clinical isolates are also MDR, which severely restricts the available treatment options, with untreatable infections in traumatic wounds often resulting in prolonged healing times, the need for extensive surgical debridement, and in some cases the further or complete amputation of limbs. Further preferred bacteria strains include G. mellonella.

One of skill in the art will appreciate that bacteria subject to the methods described herein include, but are not limited to, multidrug resistant bacterial strains. As understood herein, the terms, “multidrug resistant”, “multiple drug resistant”, “multiple drug resistance” (MDR) and like terms may be used interchangeably herein, and are familiar to one of skill in the art, i.e., a multiple drug resistant bacterium is an organism that demonstrates resistance to multiple antibacterial drugs, e.g., antibiotics.

In preferred embodiments, examples of MDR bacteria are methicillin resistant S. aureus (MRSA) and vancomycin-resistant Enterococci (VRE) vancomycin-resistant Enterococci (VRE).

As understood herein, the term “diverse sources” includes a wide variety of different places where phage may be found in the environment including, but not limited to, any place where bacteria are likely to thrive. In fact, phage are universally abundant in the environment, making the isolation of new phage very straightforward. The primary factors affecting the successful isolation of such phage are the availability of a robust collection of clinically relevant bacterial pathogens to serve as hosts, and access to diverse environmental sampling sites.

Screening methods can be employed to rapidly isolate and amplify lytic phage specific to bacterial pathogen(s) of interest to be used in generating the phage-host training set, and their therapeutic potential can be investigated. Possible sources include, e.g., natural sources in the environment such as soil, sea water, animal intestines (e.g., human intestines), as well as man-made sources such as untreated sewage water and water from waste water treatment plants. Clinical samples from infected patients may also serve as a source of phage. In one embodiment, diverse sources of phage may be selected from the group consisting of soil, water from waste water treatment plants, raw sewage, sea water, and animal and human intestines. Moreover, phage may be sourced anywhere from a variety of diverse locations around the globe, e.g., within the US and internationally. Preferably, phage can be isolated from diverse environmental sources, including soil, water treatment plants, raw sewage, sea water, lakes, rivers, streams, standing cesspools, animal and human intestines or fecal matter, organic substrates, biofilms, or medical/hospital sources.

As understood herein, the concept of “distinct and overlapping bacterial host ranges” refers to bacterial host ranges particular for a therapeutic composition. In the case of phage, the concept of “distinct and overlapping bacterial host ranges” refers to bacterial host ranges particular for a given phage, but which may overlap with the distinct host range of a different phage. For example, the concept is similar to a collection of venn diagrams; each circle can represent an individual phage's host range (or other therapeutic composition host range), which may intersect with one or more other phage's (or other therapeutic composition's) host range.

As used herein, the term “purified” refers to a preparation that is substantially free of unwanted substances in the composition, including, but not limited to biological materials e.g., toxins, such as for example, endotoxins, nucleic acids, proteins, carbohydrates, lipids, or subcellular organelles, and/or other impurities, e.g., metals or other trace elements, that might interfere with the effectiveness of the cocktail. As used herein, terms like “high titer and high purity”, and “very high titer and very high purity” refers to degrees of purity and titer that are familiar to one of skill in the art.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

### Sensitivity/Resistant Profiles of a Bacterium to a Therapeutic Composition

Determining the “bacterial host range” to a particular therapeutic molecule refers to the process of identifying the bacterial strains that are sensitive vs. resistant to the therapeutic composition. Screening to determine bacterial host range may be performed using conventional methods familiar to one of skill in the art (and as described in the examples), including but not limited to assays using robotics and other high-throughput methodologies.

Therapeutic compositions can be classified as having a broad host range (e.g., capable of having bactericidal activity on greater than 10 bacterial strains) as compared to molecules having a narrow host range (e.g., having bactericidal activity less than 5 bacterial strains). Antibiotics, for example are classified as broad vs. narrow spectrum antibiotics.

Examples of broad spectrum antibiotics for humans include, but are not limited to: Aminoglycosides (except for streptomycin), Ampicillin, Amoxicillin, Amoxicillin/clavulanic acid (Augmentin), Carbapenems (e.g. imipenem), Piperacillin/tazobactam, Quinolones (e.g. ciprofloxacin), Tetracyclines, Chloramphenicol, Ticarcillin and Trimethoprim/sulfamethoxazole (Bactrim). Examples of broad spectrum antibiotics for veterinary use, include, but are not limited to co-amoxiclav, (in small animals); penicillin & streptomycin and oxytetracycline (in farm animals); penicillin and potentiated sulfonamides (in horses).

Examples of bactericidal activities that can be considered when creating a therapeutic molecule-host sensitivity profile include lysis and/or delay in bacterial growth.

In further preferred embodiments, bactericidal activity can be measured by: (a) delay in bacterial growth of at least 0.1, at least 0.125, at least 0.15, at least 0.175, at least 0.2, or at between 0.1-0.2 OD600 absorbance difference in turbidity; (b) a lack of appearance of bacterial growth for at least 4 hours, at least 5 hours, at least 6 hours, at least 7 hours, or in between 4-6 hours; (c) reduced growth curves of surviving bacteria after treatment for at least 4 hours, at least 5 hours, at least 6 hours, at least 7 hours, or in between 4-6 hours in the Host Range Quick Test; or (d) a prevention or delay of at least 50, at least 75, at least 100, at least 125, at least 150, at least 175, at least 200, or between 50-200 relative respiration units in tetrazolium dye-based color change from active bacterial metabolism using the Omnilog bioassay of treated bacteria from the Host Range Quick Test.

In some embodiments, in building the machine learning models, the same species of bacterial pathogens can be used for the training, validation, and testing sets to train the machine learning models. In a different embodiment, different species of bacterial pathogens can be used for the training, validation and test sets for training the host machine learning models. In a further embodiment, bacterial strains comprising clinically, genotypically and/or metabolically diverse strains of the bacterial pathogen can be used to generate the machine learning models. Examples of metabolically diverse strains include, but are not limited to antibiotic resistance, ability to utilize various sugars, ability to utilize various carbon sources, ability to grow on various salts, ability to grow in presence or absence of oxygen, or bacterial motility.

In some embodiments the model identifies genomic regions in the plurality of bacterial strains as correlative of “sensitivity” vs. “resistance” to the therapeutic composition profile (“predictive patterns”), and this information can be used to predict whether a query bacterium would be sensitive vs. resistant to the therapeutic composition based on the presence or absence of the predictive patterns within the query bacterium. In one embodiment, a clinical sample is taken from a subject suffering from a bacterial infection. Typically, but not necessarily, the subject is infected with a MDR bacteria. In one embodiment, the complete genome of the query bacterium is sequenced using, preferably, rapid methods of sequencing. In some embodiment the model may explicitly output genomic regions and associates weights or parameters (the Therapeutic Composition-Host Sensitivity Sequences), and in other embodiments the information may be hidden or embodied within the model (for example in layered neural nets or classifiers). For example deep learning machine learning models (and methods), comprising multiple internal layered classifiers and/or neural nets can be utilized. In some models the genomic regions may effectively hidden within the weights and connections in the model.

Processing the sequence data as described in Block 200 results in predicting therapeutic composition-host specificity for the query bacterium. In other embodiments, rather than sequencing the entire bacterium's genome, the predictive patterns can be amplified and/or sequenced to determine the bacteria's sensitivity/resistance profile.

As used herein, the “sequencing” of the “bacterium's genome” encompasses both complete sequencing of the entire bacterium genome or the sequencing of key regions of interest that have been identified as part of the “predictive pattern”. In preferred embodiments, the complete (or substantially complete such as >99%) bacterium genome. It has been estimated that as much as 60% or 80% of genes in bacterial genomes contain genes and mechanism to defend against other phage infections. Thus preferably the complete bacterial genome is sequence, or alternatively a substantial part (eg 60%, 70%, 80%, 90% or more) in order to increase the number of genes and features that can be identified and thus used by the machine learning model to improve the predictive performance. In further preferred embodiments, gene encoding regions of the bacterium's genome is sequenced. In further preferred embodiments, genes listed in Table 1 below are sequenced. In further preferred embodiments, regions identified as comprising predictive patterns by the disclosed method are sequenced.

Once a machine learning model is generated, therapeutic compositions can be rapidly identified (by comparing the query bacterium's genome to the therapeutic composition-phage machine learning model) that would have bactericidal activity on the query bacterium. This ability to identify therapeutic composition-host profile of a query bacterium does not rely on cell culture and therefore, can be carried out rapidly, providing subject with much needed therapies in a more rapid fashion. Further as additional clinical and/or additional sequence data comes to light, the models can be retrained and refined.

### Sensitivity/Resistant Profiles of a Bacterium to a Phage

Determining the “bacterial host range” of a phage refers to the process of identifying the bacterial strains that are susceptible to infection by a given phage. The host range of a given phage is specific to a specific strain level. Screening to determine bacterial host range of a phage may be performed using conventional methods familiar to one of skill in the art (and as described in the examples), including but not limited to assays using robotics and other high-throughput methodologies.

Phage with a broad host range (e.g., capable of infecting greater than 10 bacterial strains) indicates, in general, that the receptor for said phage is common among the strains. A narrow host range (e.g., capable of infecting less than 5 bacterial strains) may indicate a unique receptor.

Determining a “phage-host sensitivity profile” of a bacterium relies on the same type of assays used to analyze a bacterial host range of a phage. Here, the goal is to screen one bacterial strain against multiple different phage to classify those phage that are able to infect and/or lyse the bacterium (a “sensitive profile”) vs. those phage that are unable to infect and/or lyse the bacterium (a “resistant profile”).

Examples of bactericidal activities that can be considered when creating a phage-host sensitivity profile include lysis, delay in bacterial growth, or a lack of appearance of phage-resistant bacterial growth. In further preferred embodiments, bactericidal activity can be measured by plaque assay. Data that can be derived from the plaque assay includes, but is not limited to: size, cloudiness and/or clarity of the plaque is measured and/or the presence of a halo around the plaque.

In further preferred embodiments, bactericidal activity can be measured by: (a) phage that can generate clear point plaques on the bacterial sample; (b) phage that demonstrate lytic characteristics using a rapid streak method on a plate; (c) bacterial lysis of at least 0.1, at least 0.2, at least 0.3, at least 0.4, at least 0.5, or between 0.1-0.5 OD600 absorbance difference in turbidity with small or large batch assays; (d) delay in bacterial growth of at least 0.1, at least 0.125, at least 0.15, at least 0.175, at least 0.2, or at between 0.1-0.2 OD600 absorbance difference in turbidity in bacteriostatic phage infections; (e) a lack of appearance of phage-resistant bacterial growth for at least 4 hours, at least 5 hours, at least 6 hours, at least 7 hours, or in between 4-6 hours post-infection; (f) reduced growth curves of surviving bacteria after phage infection for at least 4 hours, at least 5 hours, at least 6 hours, at least 7 hours, or in between 4-6 hours in the Host Range Quick Test; or (g) a prevention or delay of at least 50, at least 75, at least 100, at least 125, at least 150, at least 175, at least 200, or between 50-200 relative respiration units in tetrazolium dye-based color change from active bacterial metabolism using the Omnilog bioassay of phage-infected bacteria from the Host Range Quick Test.

In some embodiments, in building the phage-host machine learning model, the same species of bacterial pathogens can be used for the training, validation, and testing sets to train the phage-host machine learning model. In a different embodiment, different species of bacterial pathogens can be used for the training, validation and test sets for training the phage-host machine learning model. In a further embodiment, bacterial strains comprising clinically, genotypically and/or metabolically diverse strains of the bacterial pathogen can be used to generate the machine learning model. Examples of metabolically diverse strains include, but are not limited to antibiotic resistance, ability to utilize various sugars, ability to utilize various carbon sources, ability to grow on various salts, ability to grow in presence or absence of oxygen, or bacterial motility.

In some embodiments the model identifies genomic regions in the plurality of bacterial strains and/or plurality of phage strains and/or the combination thereof as correlative of “sensitivity” vs. “resistance” phage-host profile (“predictive patterns”), and this information can be used to predict whether a query bacterium would be sensitive vs. resistant to phage based on the presence or absence of the predictive patterns within the query bacterium. In one embodiment, a clinical sample is taken from a subject suffering from a bacterial infection. Typically, but not necessarily, the subject is infected with a MDR bacteria. In one embodiment, the complete genome of the query bacterium is sequenced using, preferably, rapid methods of sequencing. In some embodiment the model may explicitly output genomic regions and associates weights or parameters (the Phage-Host Sensitivity Sequences), and in other embodiments the information may be hidden or embodied within the model (for example in layered neural nets or classifiers).

Processing the sequence data as described in Block 200 results in predicting phage-host specificity for the query bacterium. In other embodiments, rather than sequencing the entire bacterium's genome, the predictive patterns can be amplified and/or sequenced to determine the infective bacteria's sensitivity/resistance profile.

As used herein, the “sequencing” of the “bacterium's genome” and/or “phage's genome” encompasses both complete sequencing of the entire bacterium/phage genome or the sequencing of key regions of interest that have been identified as part of the “predictive pattern”. In preferred embodiments, the entire genome, or at least 90%, at least 80%, at least 70%, or at least 60% of the bacterium/phage's genome is sequenced. In further preferred embodiments, gene encoding regions of the bacterium's genome is sequenced. In further preferred embodiments, genes listed in Table 1 below are sequenced. In further preferred embodiments, regions identified as comprising predictive patterns by the disclosed method are sequenced.

Once a machine learning model is generated, phage can be rapidly identified (by comparing the query bacterium's genome to the host-phage machine learning model) that would be capable of infecting and killing the query bacterium. This ability to identify phage-host profile of a query bacterium does not rely on cell culture and therefore, can be carried out rapidly, providing subject with much needed therapies in a more rapid fashion.

### Machine Learning Model

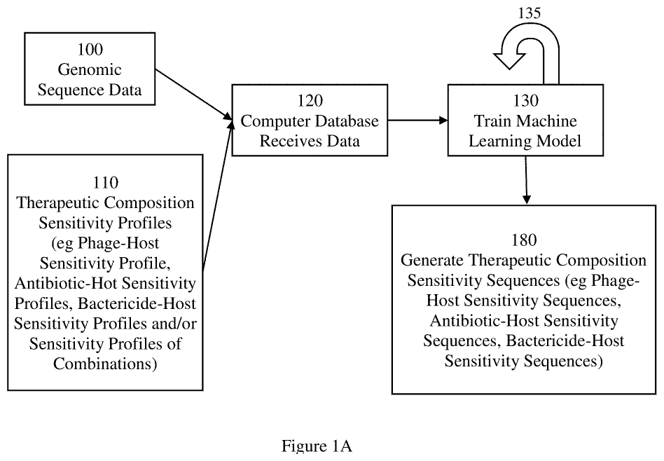

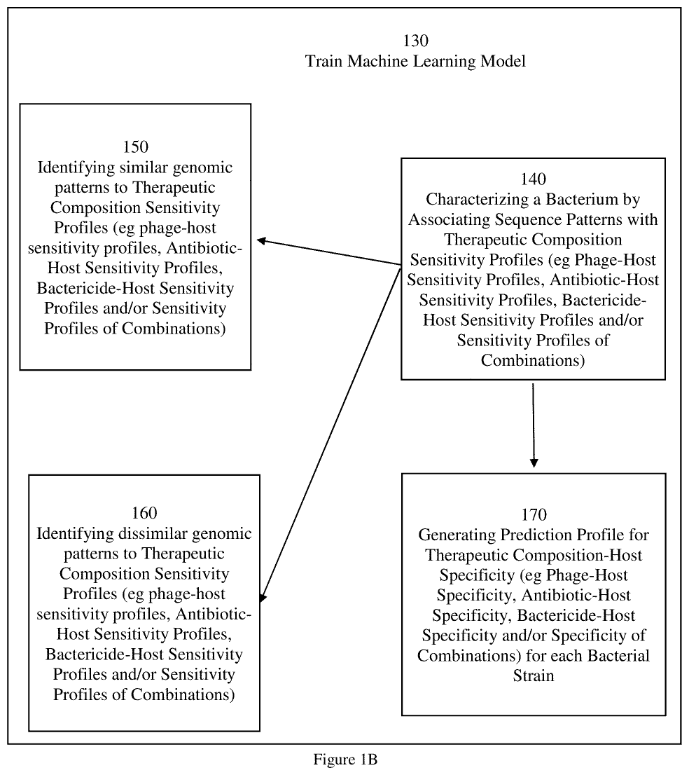

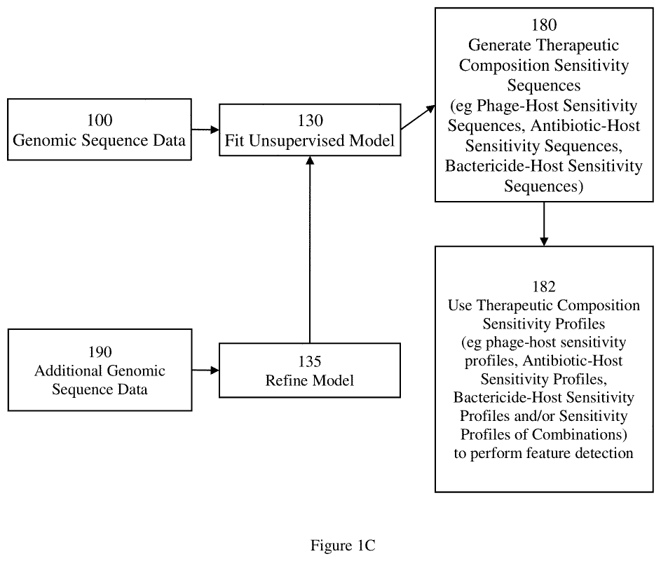

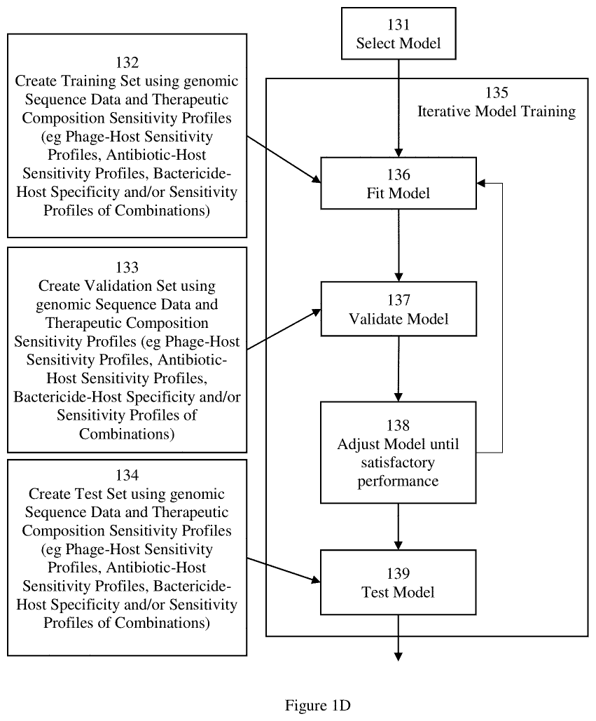

FIG. 1A illustrates one embodiment of the present invention, including an exemplary method that may be carried out by an electronic device having at least one processor and memory having instructions stored therein for carrying out the process. The method includes a computer (120) receiving genomic sequence data (100) for genomic sequence of a plurality of bacterial strains. In some embodiments the sequence data may also include genomic sequence of both a plurality of bacterial strains and a plurality of phage strains. In some embodiments therapeutic composition-host sensitivity profile data (eg phage-host sensitivity profile data, antibiotic host sensitivity profiles, bactericide-host sensitivity profiles and/or sensitivity profiles of combinations) (110) for the plurality of bacterial strains is also provided. At (130), a machine learning model is trained based on the input data (e.g., data set 100 and data set 110). In other embodiments, the training step 130 is performed iteratively, as indicated by the arrow at 135. The resulting output is a machine learning model (180) including deep learning models. This may be a computational model with human readable outputs or parameters such as a set of therapeutic composition-host sensitivity sequences (eg phage-host sensitivity sequences, antibiotic-host sensitivity sequences, and/or bactericide-host sensitivity sequences) (180) comprising sequences and weights or the computational model may be a hidden model in hidden, layered or complex model which simply generates output sensitivity estimates given an input sequence.

FIG. 1B illustrates one embodiment of how the machine learning model (130) is trained to generate prediction profiles for therapeutic composition-host specificity (eg phage-host specificity, antibiotic-host specificity, bactericide-host specificity and/or specificity of combinations) (170). Specifically, the plurality of bacterial strains are characterized (140) by associating sequence patterns with therapeutic composition-host sensitivity profiles (eg phage-host sensitivity profiles, antibiotic-host sensitivity profiles, bactericide-host sensitivity profiles and/or sensitivity profiles of combinations). This is accomplished by identifying similar and dissimilar genomic sequence patterns to similar and dissimilar therapeutic composition-host sensitivity profiles (150 and 160). At step 170, prediction profile for therapeutic composition-host specificity is outputted for each bacterial strain. However in some embodiments, rather than outputting a prediction profile for therapeutic composition-host specificity (step 170), the prediction profile is stored internally by the trained machine learning model, for example as various internal weights and model parameters.

FIG. 1C provides a flow diagram illustrating unsupervised machine learning module and updating of the model as additional data becomes available according to an embodiment. In this embodiment the genomic sequence data (100) which may be for (a) genomic sequence of a plurality of bacterial strains or (b) genomic sequence of both a plurality of bacterial strains and a plurality of phage strains is fitted using an unsupervised model. For example the data may be clustered, fit a neural network (including layered neural networks) or using latent variable models to generate phage host sensitivity sequences. In some embodiments therapeutic composition-host sensitivity profiles (eg phage-host sensitivity profiles, antibiotic-host sensitivity profiles, bactericide-host sensitivity profiles and/or sensitivity profiles of combinations) may be used to assist in feature detection (182). FIG. 1C also shows a model updating process. For example as additional genomic sequence data becomes available, this is provided to the model to refit and refine the model. For example this additional data may be the set of query bacterium of a cohort of patients obtained over an extended time (eg 12 months) since the model was last generated. The refinement of the model with additional data may be performed for any machine learning model described herein.

FIG. 1D provides a flow diagram illustrating iterative supervised machine learning involving a training set, validation set, and test set to generate a machine learning model according to an embodiment. In this example a model algorithm is first selected (eg classifier or neural net). Next a training set 132, a validation set 133 and a test set 134 each using genomic sequence data 100 and the therapeutic composition-host sensitivity profiles 110 as labels (target or outputs) are defined. A model is fitted 136 to the training set 132 using the genomic sequence data and the therapeutic composition-host sensitivity profiles 110 to determine the model weights and/or parameters that best fit the data according to some predefined criteria. The fitted model is then validated using the validation set 137, such as by providing the input validation genomic sequences and comparing the model results with the associated the phage-host sensitivity profiles. The model is then adjusted 138 (for example using backpropagation techniques) and the fitting and validation steps rerun. This iterative fitting is performed until satisfactory performance is obtained on the validation set. Once satisfactory performance is obtained the test set 134 is used to test the performance of the model 139 and the final model is saved and output performance stored.

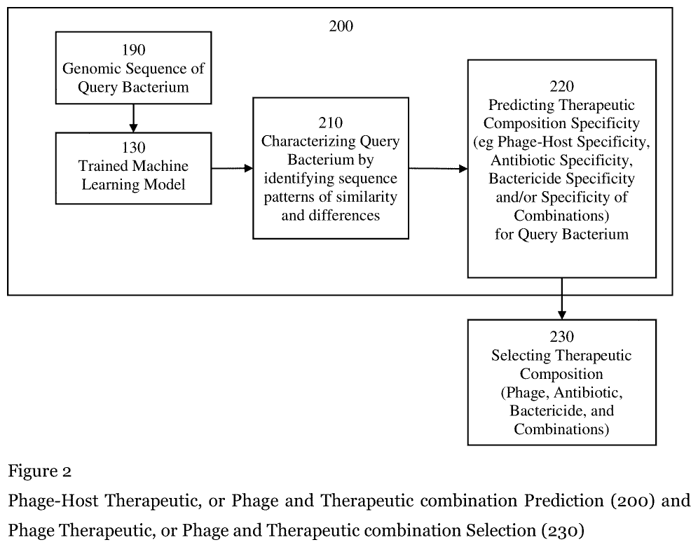

FIG. 2 illustrates how the generated machine learning model (180) can be used to make therapeutic composition-host specificity predictions (eg phage-host specificity, antibiotic specificity, bactericide specificity and/or specificity of combinations) (200) for a query bacterium as well as selection of a therapeutic composition (such as a Phage, an Antibiotic, a Bactericide, and/or a combination) (230). Genomic sequence of a query bacterium (190) is provided as input to the trained machine learning model (130). In some embodiments the machine learning model compares and processes the genomic sequence of the query bacterium (190) against the machine learning model (130) to identify similar and/or dissimilar sequence patterns as compared to the therapeutic composition-host machine learning model (210). Specificity predictions for the query bacterium are then made (220). These may be in the form of a profile match score where a higher profile match score represents a higher therapeutic composition-host sensitivity. In other embodiments sensitivity probabilities may be estimated and output for each phage-host pair. A further step can be taken to select a therapeutic composition (eg phage, an antibiotic, a bactericide, and/or a combination) identified by the process of (200) as specific to the query bacterium to be used to treat a bacterial infection or contamination. However in some embodiments the trained model may internally store therapeutic composition-host specificity information or learned common genomic sequence patterns, rather than outputting identified similarity sequences or therapeutic composition-host sensitivity sequences (step 210). In these embodiments the trained machine learning model internally processes the input genomic sequence and directly outputs predictions for the query bacterium 220. That is the genomic sequence of the query bacterium 190 is provided as input to the trained model which estimates therapeutic composition-host specificity predictions for the query bacterium (220) and exactly how the model produces these estimates is hidden or stored in a form not obvious to human inspection.

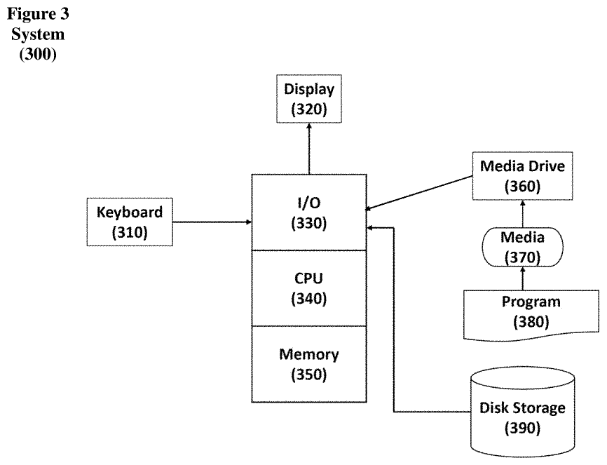

FIG. 3 depicts an exemplary computing system configured to perform any one of the processes described herein. In this context, the computing system may include, for example, a processor, memory, storage, and input/output devices (e.g., monitor, keyboard, disk drive, Internet connection, etc.). However, the computing system may include circuitry or other specialized hardware for carrying out some or all aspects of the processes. In some operational settings, the computing system may be configured as a system that includes one or more units, each of which is configured to carry out some aspects of the processes either in software, hardware, or some combination thereof. The computer system may be a distributed system including cloud based computing systems.

Specifically, FIG. 3 depicts computing system (300) with a number of components that may be used to perform the processes described herein. For example, an input/output (“I/O”) interface 330, one or more central processing units (“CPU”) (340), and a memory section (350). The I/O interface (330) is connected to input and output devices such as a display (320), a keyboard (310), a disk storage unit (390), and a media drive unit (360). The media drive unit (360) can read/write a computer-readable medium (370), which can contain programs (380) and/or data. The I/O interface may comprise a network interface and/or communications module for communicating with an equivalent communications module in another device using a predefined communications protocol (e.g. Bluetooth, Zigbee, IEEE 802.15, IEEE 802.11, TCP/IP, UDP, etc).

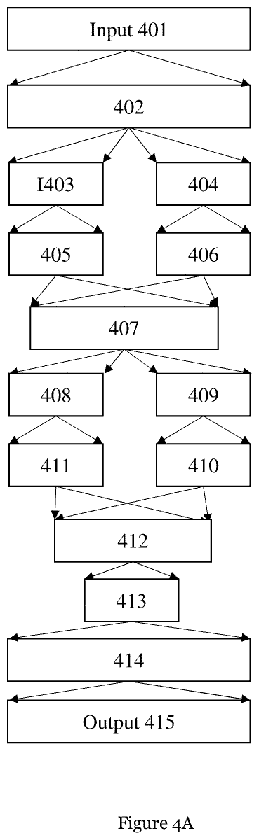

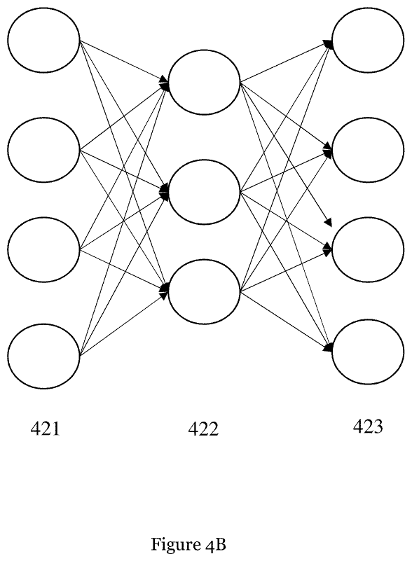

At least some values based on the results of the processes described herein can be saved for subsequent use. Additionally, a non-transitory computer-readable medium can be used to store (e.g., tangibly embody) one or more computer programs for performing any one of the above-described processes by means of a computer. The computer program may be written, for example, in a general-purpose programming language (e.g., Pascal, C, C++, Java, Python, JSON, Perl, MATLAB, R, etc.) or some specialized application-specific language. A range of machine learning and deep learning software libraries such as TensorFlow, scikit-learn, Theano, Apache Spark MLlib, Amazon Machine Learning, Deeplearning4j, etc, can also be used. FIG. 4A illustrates an exemplary architecture of deep learning model comprising multiple internal layers (402 to 414) for use in the method and systems described herein. For example the deep learning model could be a convolution neural network model with an input layer 401, and a set of convolution filter with rectifier linear units (ReLU) activation, also known as rectifier activation functions 402 to 414, and an output layer 415. In other embodiments, other deep learning models as described above could be used. FIG. 4B illustrates connections between neurons in layers in a deep learning model for use in the method and systems described herein. For example a first set of neurons in a first layer 421 are connected to a second set of neurons in a second layer 422. These in turn are connected to a third set of neurons in a third layer 423. Weights are applied on each connection (ie to each arrow). In the training process, the inputs are processed by the model, and a loss (or cost or error) function estimates performance, such as by comparing the prediction to a known result (supervised learning) or benchmark. The weights on the individual layers can then be altered, for example using backpropagation techniques, and the input reprocessed and the loss function recalculated. This training process is repeated until acceptable performance is achieved. Further as additional data is obtained (eg clinical results from use of a specific phage or therapeutic composition against a specific bacteria), the model can be refined and retrained.

Also provided is a non-transitory computer-readable storage medium comprising computer-executable instructions for carrying out any of the methods described herein. Further provided is a computer system comprising one or more processors, memory, and one or more programs, wherein the one or more programs are stored in the memory and configured to be executed by the one or more processors, the one or more programs including instructions for carrying out any of the methods described herein.

### Compositions and Methods of Treatment

In another aspect, the instant invention relates to therapeutic compositions (“cocktails”) comprising phage, antibiotics, and/or bactericides (including a mixture of phage, antibiotics and/or bactericides) identified in the process described as Block 200. In a particular embodiment, the compositions are therapeutically effective phage cocktails of very high titer and purity which are not found in nature. Moreover, while the methods described herein may be used to formulate a personalized phage cocktail directed to a subject's particular bacterial infection, it is contemplated herein that the cocktail could be used to treat other individuals infected with the same or very similar bacterial strain(s) with patterns of infectivity as recognized and defined by the machine learning system. Thus, the method may be used to generate phage cocktails that have broad therapeutic use.

Moreover, and in another aspect, the instant invention relates to therapeutic compositions comprising therapeutic molecules, such as antibiotics or other bactericidal compounds (including mixtures) identified in the process described as Block 200. In preferred embodiments, the therapeutic composition work synergistically with one another—such as for example, a phage cocktail administered in combination with one or more antibiotics and/or other bactericidal compounds.

As understood by one of skill in the art, the type and amount of pharmaceutically acceptable additional components included in the pharmaceutical compositions may vary, e.g., depending upon the desired route of administration and desired physical state, solubility, stability, and rate of in vivo release of the composition.

As contemplated herein, the phage cocktails, and particularly pharmaceutical compositions of the phage cocktails, comprise an amount of phage in a unit of weight or volume suitable for administration to a subject. The volume of the composition administered to a subject (dosage unit) will depend on the method of administration and is discernible by one of skill in the art. For example, in the case of an injectable, the volume administered typically may be between 0.1 and 1.0 ml, e.g., approximately 0.5 ml, with a maximal permissible level of endotoxin in injected products is 5 EU/kg/hour or 350 EU/hour in a 70 kg person.

For administration by intravenous, cutaneous, subcutaneous, or other injection, a pharmaceutical formulation is typically in the form of a parenterally acceptable aqueous solution of suitable pH and stability, and may contain an isotonic vehicle as well as pharmaceutical acceptable stabilizers, preservatives, buffers, antioxidants, or other additives familiar to one of skill in the art.

### Methods of Treatment

The therapeutic compositions, such as for example, phage cocktails, generated according to the methods of the invention can be used to treat a bacterial infection in a subject or bacterial contamination in the environment. Such methods of treatment include administering to a subject in need thereof an effective amount of a the composition (e.g., phage cocktail) described herein.

It will be appreciated that appropriate dosages of the active compounds or agents can vary from patient to patient. Determining the optimal dosage will generally involve the balancing of the level of therapeutic benefit against any risk or deleterious side effects of the administration. The selected dosage level will depend on a variety of factors including, but not limited to, the route of administration, the time of administration, the rate of excretion of the active compound, other drugs, compounds, and/or materials used in combination, and the age, sex, weight, condition, general health, and prior medical history of the patient. The number of active compounds and route of administration will ultimately be at the discretion of the physician, although generally the dosage will be to achieve concentrations of the active compound at a site of therapy without causing substantial harmful or deleterious side-effects.

In general, a suitable dose of the active compound or agent is in the range of about about 1 μg or less to about 100 μg or more per kg body weight. As a general guide, a suitable amount of a phage cocktail of the invention can be an amount between from about 0.1 μg to about 10 mg per dosage amount.

In addition, the therapeutic compositions, including a phage cocktail and/or in combination with one or more antibiotics, described herein can be administered in a variety of dosage forms. These include, e.g., liquid preparations and suspensions, including, preparations for parenteral, subcutaneous, intradermal, intramuscular, intraperitoneal, intra-nasal (e.g., aerosol) or intravenous administration (e.g., injectable administration), such as sterile isotonic aqueous solutions, suspensions, emulsions or viscous compositions that may be buffered to a selected pH. In a particular embodiment, it is contemplated herein that the phage cocktail is administered to a subject as an injectable, including but not limited to injectable compositions for delivery by intramuscular, intravenous, subcutaneous, or transdermal injection. Such compositions may be formulated using a variety of pharmaceutical excipients, carriers or diluents familiar to one of skill in the art.

In another particular embodiment, the therapeutic composition, including the phage cocktail described herein, may be administered orally. Oral formulations for administration according to the methods of the present invention may include a variety of dosage forms, e.g., solutions, powders, suspensions, tablets, pills, capsules, caplets, sustained release formulations, or preparations which are time-released or which have a liquid filling, e.g., gelatin covered liquid, whereby the gelatin is dissolved in the stomach for delivery to the gut. Such formulations may include a variety of pharmaceutically acceptable excipients described herein, including but not limited to mannitol, lactose, starch, magnesium stearate, sodium saccharine, cellulose, and magnesium carbonate.

In a particular embodiment, it is contemplated herein that a composition for oral administration may be a liquid formulation. Such formulations may comprise a pharmaceutically acceptable thickening agent which can create a composition with enhanced viscosity which facilitates mucosal delivery of the active agent, e.g., by providing extended contact with the lining of the stomach. Such viscous compositions may be made by one of skill in the art employing conventional methods and employing pharmaceutical excipients and reagents, e.g., methylcellulose, xanthan gum, carboxymethyl cellulose, hydroxypropyl cellulose, and carbomer.

Other dosage forms suitable for nasal or respiratory (mucosal) administration, e.g., in the form of a squeeze spray dispenser, pump dispenser or aerosol dispenser, are contemplated herein. Dosage forms suitable for rectal or vaginal delivery are also contemplated herein. The constructs, conjugates, and compositions of the instant invention may also be lyophilized and may be delivered to a subject with or without rehydration using conventional methods.

As understood herein, the methods of administering a therapeutic composition, including a phage cocktail described herein and/or in combination with an antibiotic or other bactericidal compound, to a subject can occur via different regimens, i.e., in an amount and in a manner and for a time sufficient to provide a clinically meaningful benefit to the subject. Suitable administration regimens for use with the instant invention may be determined by one of skill in the art according to conventional methods. For example, it is contemplated herein that an effective amount may be administered to a subject as a single dose, a series of multiple doses administered over a period of days, or a single dose followed by a boosting dose thereafter.

The administrative regimen, e.g., the quantity to be administered, the number of treatments, and effective amount per unit dose, etc. will depend on the judgment of the practitioner and are subject dependent. Factors to be considered in this regard include physical and clinical state of the subject, route of administration, intended goal of treatment, as well as the potency, stability, and toxicity of the therapeutic compositions, including a phage cocktail. As understood by one of skill in the art, a “boosting dose” may comprise the same dosage amount as the initial dosage, or a different dosage amount. Indeed, when a series of doses are administered in order to produce a desired response in the subject, one of skill in the art will appreciate that in that case, an “effective amount” may encompass more than one administered dosage amount.

Although the invention herein has been described with reference to embodiments, it is to be understood that these embodiments, and examples provided herein, are merely illustrative of the principles and applications of the present invention. It is therefore to be understood that numerous modifications can be made to the illustrative embodiments and examples, and that other arrangements can be devised without departing from the spirit and scope of the present invention as defined by the appended claims. All patent applications, patents, literature and references cited herein are hereby incorporated by reference in their entirety.

### EXAMPLES

The invention will now be further illustrated with reference to the following examples. It will be appreciated that what follows is by way of example only and that modifications to detail may be made while still falling within the scope of the invention.

### Example 1: Phage Isolation/Characterization from Environmental Sources

Powdered TSB medium (Becton, Dickinson and Company) can be mixed with raw sewage to a final concentration of 3% w/v. Different bacterial strains can be grown to exponential phase, and 1 mL of each strain added to 100 mL aliquots of TSB-sewage mixture, and incubated at 37° C. and 250 rpm overnight. The following day, 1 mL of the infected TSB-sewage mixture is harvested and centrifuged at 8,000×g for 5 min to pellet cells and debris. The supernatant is transferred to a sterile 0.22 μm Spin-X® centrifuge tube filter (Coming, N.Y.), and centrifuged at 6,000×g to remove any remaining bacteria. A 10 μL aliquot of the filtrate is mixed with 100 μL of exponential growth culture of the bacterial strain, incubated at 37° C. for 20 min, mixed with 2.5 mL of molten top agar (0.6% agar) tempered to 50° C., and poured over TSB agar plates (1.5% TSB agar). Plates are incubated overnight at 37° C., and subsequent phage plaques are individually harvested and purified three times on appropriate bacterial strain isolates using the standard procedures described by, for example, Sambrook et al.

If desired, high-titer phage stocks can be propagated and amplified in corresponding host bacteria by standard procedures known to the skilled artisan. Large-scale phage preparations can be purified by caesium chloride density centrifugation, and filtered through a 0.22 μm filter (Millipore Corporation, Billerica, Mass.).

For example, phage can be purified by Caesium chloride gradient as is well known in the art. Here, the generated purified phage suspension (1 ml) can be precipitated with 10% polyethylene glycol 8000 (Sigma-Aldrich) and 0.5 M sodium chloride at 4° C. overnight. Subsequently, the suspension can be centrifuged at 17,700 g for 15 minutes and the supernatant removed. Alternatively, the phage suspension can be dialyzed. The PEG/salt-induced precipitate is resuspended in 0.5 ml of TE buffer (pH 9.0) and treated with 20 ul of 20 mg/ml proteinase K for 20 minutes at 56° C. followed by treatment with SDS at a final concentration of 2% at 65° C. for 20 minutes. This mixture is then phenol/chloroform (25:24:1 phenol:chloroform:isoamyl alcohol, Sigma Aldrich) treated at least twice and the aqueous phase is then precipitated with 2.5 volumes of ice cold 96% ethanol and 0.1 volume of sodium acetate (pH 4.8). Subsequent to centrifugation, the pellet is washed in 70% ethanol and resuspended in 100 ul of TE buffer (pH 8.0). Phage stocks can then be stored at 4° C. indefinitely. Phage titer can be assessed by plating ten-fold serial dilutions and calculating the plaque forming units (PFU).

Other methods of phage purification include, but are not limited to partition separations with either octanol or butanol. In this technique, phage normally stay in the aqueous phase while endotoxins tend to be absorbed by the alcohol phase.

### Example 2: Assays Used to Generate Phage-Host Sensitivity Profiles

To carry out the disclosed method, the genomes of multiple different bacterial strains having similar or identical phage-host sensitivity profiles need to be compared. If a phage-host sensitivity profile of a bacterium is already known, the following assays do not need to be performed. However, if the phage-host sensitivity profile of a bacterium is unknown, any of the following assays can be used to determine or experimentally derive such a profile.

One method of determining a sensitivity/resistant profile of a bacterium relies on an automated, indirect, liquid lysis assay. Briefly, an overnight culture of a bacterial strain is inoculated into the wells of a 96-well plate containing TSB mixed with 1% v/v tetrazolium dye. Phage are then added to each well, and plates were incubated in an OmniLog™ system (Biolog, InC, Hayward, Calif.) at 37° C. overnight. See, Henry, Bacteriophage 2:3, 159-167 (2012). The tetrazolium dye indirectly measures the respiration of the bacterial cells. Respiration causes reduction of the tetrazolium dye, resulting in a color change to purple. The color intensity of each well is quantified as relative units of bacterial growth. For host range determination, bacteria are inoculated at 10⁵ colony forming units (CFU) per well and phage are added at a concentration of 10⁶ plaque forming units (PFU) per well for an MOI of 10. For cocktail synergy studies, bacteria can be inoculated at 10⁶ CFU per well and phage added at a concentration of 10⁸ PFU per well for an MOI of 100.

A second assay can also be used to determine the sensitivity/resistant profile of a bacterium. In this assay, a dilution series spot plate assay is used to observe plaque formation. Specifically, 50 μL of an overnight culture of a bacterium is used to individually inoculate 5 mL of molten top agar tempered to 55° C. The inoculated agar is then mixed thoroughly by brief vortexing and then spread over square LB agar plates. Top agar is allowed to set for approximately 45 min, at which time 4 μL aliquots of 10¹⁰ to 10² PFU in to-fold dilutions of each phage are spotted on the surface. Spots are allowed to fully absorb into the top agar, after which plates were incubated at 37° C. for 24 hours. Plaque formation can then be assessed.

Time-kill experiments can also be used to provide a quantitative sensitivity/resistant profile of a bacterium. Here, an overnight culture of a bacterium is diluted 1:1000 in fresh LB broth to a final concentration of approximately 1×10⁶ CFU per mL. Twenty mL aliquots are then transferred to 250 mL Erlenmeyer flasks and incubated at 37° C. with shaking at 200 rpm for 2 hours. Samples are then challenged with either 2×10¹¹ PFU per mL of a phage or an equal volume of sterile phosphate buffered saline (PBS) and returned to incubation. One hundred μL aliquots are taken at 0, 2, 4, and 24 hours, serially diluted in PBS, and plated on LB agar. Plates are incubated at 37° C. for 24 hours and plaque formation is evaluated.

Changes in a bacterium due to phage exposure can also be monitored using Raman spectroscopy. Here, each sample is obtained from LB agar plates and are directly transferred into a disposable weigh dish for spectral collection. Raman spectra can be collected using an 830 nm Raman PhA T system (Kaiser Optical Systems, InC, Ann Arbor, Mich., USA). Spectra are collected using a 3 mm spot size lens with 100 sec total acquisition time and 1 mm spot size lens with 100 sec total acquisition time for time-kill assay samples. Spectra are then preprocessed by baseline removal using a sixth order polynomial and intensity normalization to the 1445 cm⁻¹ Raman vibrational band prior to analysis.

Other examples of bactericidal activities that can be considered when creating a sensitivity/resistant profile include lysis, delay in bacterial growth, or a lack of appearance of phage-resistant bacterial growth. In further preferred embodiments, bactericidal activity can be measured by: (a) phage that can generate clear point plaques on the bacterial sample; (b) phage that demonstrate lytic characteristics using a rapid streak method on a plate; (c) bacterial lysis of at least 0.1, at least 0.2, at least 0.3, at least 0.4, at least 0.5, or between 0.1-0.5 OD600 absorbance difference in turbidity with small or large batch assays; (d) delay in bacterial growth of at least 0.1, at least 0.125, at least 0.15, at least 0.175, at least 0.2, or at between 0.1-0.2 OD600 absorbance difference in turbidity in bacteriostatic phage infections; (e) a lack of appearance of phage-resistant bacterial growth for at least 4 hours, at least 5 hours, at least 6 hours, at least 7 hours, or in between 4-6 hours post-infection; (f) reduced growth curves of surviving bacteria after phage infection for at least 4 hours, at least 5 hours, at least 6 hours, at least 7 hours, or in between 4-6 hours in the Host Range Quick Test; or (g) a prevention or delay of at least 50, at least 75, at least 100, at least 125, at least 150, at least 175, at least 200, or between 50-200 relative respiration units in tetrazolium dye-based color change from active bacterial metabolism using the Omnilog bioassay of phage-infected bacteria from the Host Range Quick Test.

Using these assays, one can test multiple phage against a diverse set of bacterial strains to create a phage-host sensitivity profile. This profile can be based on both the ability of the phage to infect a bacterium, and also could be based, for example, on the number of hours each phage could prevent growth of the bacterial host in liquid (hold time) and/or the clarity/turbidity of the plaque. Once a phage-host sensitivity profile is experimentally generated for multiple bacterial strains, the strains can be categorized into groups exhibiting similar profiles using the processes as described herein.

The assays described in this example can readily be modified to screen for other bactericidal compounds to be used as described herein.

### Example 3: Genome Sequencing, Assembly & Annotation

Phage and/or bacteria genomes can be sequenced using standard sequencing techniques and assembled using contig analysis as is well known in the art. For example, 5 ug of DNA isolated from phage or bacteria can be extracted and shipped to a contract sequencing facility. A 40- to 65-fold sequencing coverage is obtained using pyrosequencing technology on a 454 FLX instrument. The files generated by the 454 FLX instrument are assembled with GS assembler (454, Branford, Conn.) to generate a consensus sequence. Quality improvement of the genome sequence can involve sequencing of 15-25 PCR products across the entire genomes to ensure correct assembly, double stranding and the resolution of any remaining base-conflicts occurring within homopolynucleotide tracts. Protein-encoding open reading frames (ORFs) can be predicted using standard programs known in the art (such as BLASTP) followed by manual assessment and, where necessary, correction.

### Example 4: Prediction of a Query Bacterium's Phage-Host Profile

Although Examples 4 and 5 are directed to identifying patterns of nucleotide sequences between bacteria and phage, the same approach can readily be modified to identify genomic patterns bacteria that reflect sensitivity vs. resistance to any therapeutic composition, such as an antibiotic and/or other bactericidal compound (or therapeutic molecule).

The disclosed methods are based on the capacity of computer AI Neural network analysis to discover genomic sequence patterns to facilitate the recognition of specific phage that have the capacity of that phage to serve as an effective antibacterial agent for a clinically isolated bacterial strain. To accomplish this goal, we are using machine learning, such as Neural network analysis, to search the patterns of nucleotide sequences in the genomes of (a) bacteria or (b) bacteria and phages to predict whether or not a specific phage can act as an antibacterial agent by killing, replicating in, lysing, or inhibiting the growth of the clinical isolated bacterial strain. Such computer-based predictions would significantly reduce the time required to find a phage for the treatment of an infection.

This approach differs from earlier efforts which use computers to find associations between phages that affect certain strains of bacteria or vis versa. The prior approaches searched for known offensive and defensive phage and bacterial systems, including phage receptor sites on bacterial strains or, on matches based on nucleotide homologies (refs 6-7). However, it is unlikely that such matches will provide reliable clinical guidance due to the complexity of the interactions between the offensive and defensive tools that have been developed over the course of 4 billion years of phage-bacterial interactions (references 1-7).

The discovery of mechanisms that bacteria have evolved to protect themselves against phage and the counter-measures developed by phage is currently a subject of intense research activity. The mechanisms uncovered thus far are numerous and often complex. They include the recently elucidated phage use of specific proteins to defend against the clustered regularly interspaced short palindromic repeat (CRISPR)-Cas phage immunity mechanisms. For example, Table 1 reproduced below from the review by Sampson et al., “Revenge of The Phages: Defeating Bacterial Defenses” Nat. Rev. Microbiol. 11: 675-687, 2013 outlines some of the bacterial defenses and the phage mechanisms that have evolved to overcome them.

TABLE 1
Summary of the strategies used by phages to by-pass bacterial antiviral systems

| | | | |
|---|---|---|---|
| Phage escape strategy | Phages | By-pass mechanism | Refs |

Inhibition of phage adsorption

| | | | |
|---|---|---|---|
| Adapting to new | Coliphage λ | A modified RBP recognizes a new receptor | 12 |
| receptors | Coliphages ϕX174 and T7 | Mutations in the RBP-encoding gene lead to adsorption to a | 13, 14, |
| | | modified LPS structure | 16 |
| | Lactobacillus delbrueckii subsp. lactis | Modified tail proteins bind new receptors | 17-20 |
| | phage LL H, Prochlorococcus phages | | |
| | and Pseudomonas fluorescens phage ϕ2 | | |
| Digging for | Coliphages K1F and K1-5 | An encoded endosialidase or glycosidase degrades the | 21, 22 |
| receptors | | Escherichia coli polysaccharide capsule | |
| | Streptococcus pyogenes phage H4489A | An encoded hyaluronan lyase hydrolyses the hyaluronic acid | 24 |
| | | capsule | |
| | Pseudomonas spp. phages | EPS-degrading enzymes break down EPS to allow the RBP to | 25, 26 |
| | | access the receptor | |
| Stochastic | Bordetella spp. phages | Mutations in mtd lead to adsorption to phase-variable | 28-30 |
| recognition | | receptors | |
| of variable | Lactococcus lactis phages TP901-1 | Tail-associated peptidases increase phage adsorption to | 31 |
| host receptors | and Tuc2009 | hosts with a variable physiological state (exponential versus | |
| | | stationary phase cells) | |
| | Coliphage T4 | Duplication of a His box element in the tail proteins leads to | 32 |
| | | shuffling of the tail specificity for host receptors | |

Restriction-modification (R-M) systems

| | | | |
|---|---|---|---|
| Fewer restrictions | Coliphages T7 and T3 | EcoRII recognition sites in the genome are distant from each | 41 |
| sites or sites in a | | other; which prevents cleavage by the REase EcoRII | |
| non-recognizable | Coliphage T7 | EcoP15I recognition sites are oriented in the same direction | 42 |
| orientation | | and thus avoid being cut by REase | |
| Modification of | Bacillus spp. phages | Uracil or hydroxymethyl uracil replacing thymine in | 39, 43 |
| restriction sites | | the genome prevents recognition and cleavage of | |
| | | thymine-containing sites by REases | |
| | T-even coliphages | Hydroxymethyl cytosine and/or glucosylated hydroxymethyl | 39 |
| | | cytosine in the DNA is not recognized by REases that use | |
| | | cytosine in the recognition site | |
| | Mu-like phages | Mom or its derivatives modify adenine to N′-adenine, | 52, 53 |
| | | protecting adenine-containing restriction sites against REase | |
| | | activity | |
| Masking restriction sites | Coliphage P1 | DarA and Dar8 are co-injected into the host cell and bind to | 47 |
| or mimicry of phage DNA | | restriction sites in the phage genome, protecting it against | |
| | | type IR-M systems | |
| | Coliphage T7 | Ocr mimics DNA backbone and has a high affinity for the | 48, 49 |
| | | EcoKI REase component; thereby sequestering the REese | |
| Stimulation of | Coliphage λ | Ral enhances the activity of the EcoKI methyltransferase | 54-56 |
| the modification | | component, resulting in rapid methylation of phage DNA, | |
| enzyme | | which is thereby protected from recognition by the EcoKI REase | |
| | Coliphage T4 | The peptide Stp disrupts the structural conformation of the | 57 |
| | | Ecoprrl system | |
| Degradation of an | Coliphage T3 | An encoded S-adenosyl-L-methionine hydrolase removes | 58 |
| R-M cofactor | | S-adenosyl-L-methionine (an essential R-M cofactor), thereby | |

CRISPR-Cas systems

| | | | |
|---|---|---|---|
| Mutation in the | Streptococcus thermophilus phages | Mutations or deletions in the protospacer or PAM results in | 62 |
| protospacer or | | avoidance of CRISPR-Cas systems | |
| in the PAM | | | |
| Anti-CRISP | Pseudomonas aeruginosa prophages | Anti-CRISPR genes encode proteins that interfer with the host | 68 |
| proteins | | CRISPR-Cas system | |
| Antibacterial | Vibrio cholerae serogroup O1 ICP1 phages | A phage-encoded CRISPR-Cas system targets another | 70 |
| CRISPR-Cas systems | | bacterial antiphage mechanism found on a phage-inducible | |
| | | chromosomal island in the host genome | |

Abortive-infection (Abi) systems

| | | | |
|---|---|---|---|
| Mutations in | Coliphage T4rll | Mutation of motA allows the phage to avoid Rex-mediated | 73, 74, |
| specific phage | | exclusion | 77 |
| genes | Coliphage T4 | Mutation of the gene encoding the peptide Gol prevents | 80, 82 |
| | | activation of the Abi system Lit | |
| | Lactococcus spp. phages | Mutations in different genes allow evasion of Abi systems such | 85, 90, |
| | | as AbiD1, AbiK, AbiV, AbiT and AbiO | 94, 96, |
| | | | 109 |
| | Lactococcus spp. phages | The exchange of large genomic regions with resident | 89, 91 |
| | | prophages allows virulent phages to escape phage resistance | |
| | | systems | |
| Encoding an | Coliphage T4 | Dmd neutralizes the toxic effect of the toxins RnlA and LsoA | 103 |
| antitoxin | | during phage replication | |
| molecule | Pectobacterium atrosepticum phage ϕTE | A pseudo-antitoxin mimic or hijacking the host antitoxin | 107 |
| | | protects the phage against the ToxIN system | |

Cas, CRISPR-associated proteins; CPISPR, clustered regularly interspaced short palindromic repeats; EPS, exopolysacchadde; LPS, lipopolysaccharide: mtd, major tropism determinant; PAM, protospacer-adjacent motif; RBP, receptor-binding protein; REase, restriction endonuclease.

Given that there are an estimated 10³¹ phage on the Earth and 10³⁰ bacteria, Table 1 is only an early beginning for our delineation of the bacterial arsenal of defense mechanisms, and phage responses to these mechanisms. Even at this level of discovery we need to be aware that bacterial defense is often multilayered, and each bacterial strain may incorporate more than one of these defense mechanisms.

For an example of a type of interaction. that cannot be delineated in current genomic “matching searches”, involves certain strains of E. coil that have evolved specific polysaccharide outer capsules that can serve as a barrier to phage infection. To counter this defense, phages have evolved proteins (enzymes) attached to their tail fibers that have the capacity to digest specific polysaccharide outer capsules. However, a different enzyme is required to “digest” each of the specific polysaccharide outer capsules that have evolved to protect a bacterial “host”. Genomic “matching searches” based. on homologies between the bacterial and viral sequences are unlikely to recognize a gene for a specific phage protein that codes for an enzyme that can degrade a bacterial polysaccharide outer capsule that is synthesized in the bacteria by a number of enzymes, and certainly it would not, at this time, be possible to predict the specificity for specific polysaccharide of the phage enzyme from the sequence data for the phage enzyme (see references by Scholl et al., references: 8-10).

Thus, the limitations noted for the strategies based on known offensive and defensive phage-bacterial systems or on matches based on nucleotide homologies can be overcome by the methods described herein using machine learning/artificial computer intelligence to search for predicative patterns in the nucleotide sequences of (a) bacteria or (b) bacteria and phage genomes, or to otherwise classify or identify phage-host specificity based on sequence data.

For example, use of computer “Neural network analysis” or deep learning approaches to search the genomes of the phage and their “host” bacteria, in a manner analogous to the “Deep Mind” methods Google used to develop computer-based translation and game playing programs combined with Bayesian machine learning, as employed by the IBM “Watson” system. Discovery of such patterns can be facilitated by “training” the computer to “recognize” nucleotide patterns in specific phage strains that have proven to be effective as antibacterial agents against the nucleotide patterns of clinical bacterial isolates that have been proven to be susceptible to those phage strains. Such an effort will also require training computer systems with bacteria that are resistant to specific phage strains.

The development of “Neural network analysis” or other machine learning platforms to search for predicative patterns in the nucleotide sequences as described herein of both phage and bacterial host genomes may provide the major pathway that satisfied an unmet need for rapid ways to predict a bacterial strains' sensitivity to specific phage strains for clinical/environmental applications without the need to perform cell-kill curves. Given “sufficient” training with such data sets it is expected that the artificial computer intelligence system will be able to predict which phage can successfully infect a specific bacterium—based on the provided genome sequence data—as well as those that are resistant to infection.

### REFERENCES

1. Intriguing arms race between phages and hosts and implications for better anti-infectives. Zhang Z, Huang C, Pan W, Xie J. Crit Rev Eukaryot Gene Expr. 2013; 23(3):215-26.

2. Revenge of the phages: defeating bacterial defenses. Samson J E, Magadán A H, Sabri M, Moineau S. Nat Rev Microbiol. 2013 October; 11(10):675-687

3. Bacteriophage resistance mechanisms. Labrie S J, Samson J E, Moineau S. Nat Rev Microbiol. 2010 May; 8(5):317-327.

4. Molecular mechanisms of CRISPR-mediated microbial immunity. Gasiunas G, Sinkunas T, Siksnys V., Cell Mol Life Sci. 2014 February; 71(3):449-65.

5. Inhibition of CRISPR-Cas9 with Bacteriophage Proteins, Rauch, B. J., Melanie, R. S., Judd, F. H., Christopher, S. W., Michael, J. M., Nevan, J. K., and Joseph, B-D., Cell 168: 1-9, Jan. 12, 2017

6. Computational approaches to predict bacteriophage-host relationships, Edwards, R. A., Katelyn McNair, K., Faust, K., Raes, J., and Dutilh, B. E., FEMS Microbiology Reviews, fuvo48, 40: 258-272, 2016.

7. HostPhinder: A Phage Host Prediction Tool, Villarroel, J., Kleinheinz, K. A., Jurtz, V. I., Zschach, H., Lund, O., Nielsen, M., and Larsen, M., V., Viruses 8: 116-138, 2016

8. Scholl, D., Adhya, S., and Merril, C. R., The E. coli K1 capsule acts as a barrier to phage T7. In: Applied And Environmental Microbiology, 71: 4872-4874, 2005

9. Scholl, D., and Merril, C. R., Polysaccharide Degrading Phages (Eds. Waldor, M. K., Friedman, D. I. and Adhya, S. L.) In: Phage: Their role in Bacterial Pathogenesis and Biotechnology, American Society of Microbiology 400-414, 2005.

10. Scholl, D., and Merril, C. R., The Genome of Bacteriophage K1F, a T7-Like Phage That Has Acquired the Ability To Replicate on K1 Strains of Escherichia coli, Journal of Bacteriology, 187: 8499-8503, 2005.

11. Artificial Neural Network Prediction of Viruses in Shellfishs, Brion G., Viswanathan C, Neelakantan T R, Lingireddy S, Girones R, Lees D, Allard A, Vantarakis A., Appl Environ Microbiol. September;71(9):5244-5253. 2005

### Example 5: Amplification of Predictive Regions by Multiplex PCR

In this example, the genomic sequences encompassing the predictive regions identified thru the method described herein for a phage-host sensitivity profile can be analyzed as described in Block 130. Using this data, primers can be designed to amplify these predictive regions along with a control. The multiplex PCR can include different sets of primers and then applied to the strains assessed in the host range analysis under the following conditions: 95° C. for 6 minutes followed by 31 cycles of 95° C. for 15 seconds, 570 C for 30 seconds and 72° C. for 1 minute and a final extension step at 72° C. for 7 minutes.

The invention is not limited to the embodiment herein before described which may be varied in construction and detail without departing from the spirit of the invention. The entire teachings of any patents, patent applications or other publications referred to herein are incorporated by reference herein as if fully set forth herein.

1. A computational method for generating a therapeutic composition machine learning model, wherein the method comprises:
(a) compiling data from a plurality of bacterial strains in a computer database system, wherein the data comprises genomic sequence data of a plurality of bacterial strains;
(b) training a machine learning model using at least the genomic sequence data of a plurality of bacterial strains on a CPU and a memory unit of a computer system; and
(c) storing a therapeutic composition machine learning model configured to receive a query bacterial genome and select at least one therapeutic composition estimated to be sensitive to the bacterial genome based on the trained machine learning model.

2. The method as claimed in claim 2, wherein the at least one therapeutic composition estimated to be sensitive to the bacterial genome based on the trained machine learning model comprises one or more phage, antibiotic, bactericide, therapeutic molecule or combination estimated to be sensitive to the bacterial genome based on the trained machine learning model.

3. The computational method of claim 2, wherein the least one therapeutic composition comprises at least one phage and,
in step (a) the data further comprises:
genomic sequence data of a plurality of phage strains;
and in step (b) training a machine learning model uses at least the genomic sequence data of a plurality of bacterial strains and the genomic sequence data of a plurality of phage strains on a CPU and a memory unit of a computer system; and
in step (c) the therapeutic composition machine learning model configured to receive a query bacterial genome is configured to select at least one phage estimated to be sensitive to the bacterial genome based on the trained machine learning model.

4. The method as claimed in claim 1, wherein the machine learning model generates therapeutic composition sensitivity sequences.

5. The method as claimed in claim 4, further comprising receiving experimentally derived therapeutic composition-host sensitivity profiles of the bacterial strains experimentally derived from a plurality of therapeutics, and generating the therapeutic composition sensitivity sequences comprises performing feature detection using the therapeutic composition-host sensitivity profiles comprising:
(1) identifying common genomic sequence patterns shared between the bacterial strains having similar or identical therapeutic composition-host sensitivity profiles; and/or
(2) identifying dissimilar genomic sequence patterns shared between the bacterial strains having dissimilar therapeutic composition-host sensitivity profiles;
and training the model further comprises characterizing each bacterial strain by associating the therapeutic composition Sensitivity Sequences with therapeutic composition-host sensitivity profiles and generating a prediction profile for therapeutic composition-host specificity for each bacterial strain.

6. The method as claimed in claim 5, further comprising receiving additional genomic sequence data and therapeutic composition-host sensitivity profiles for a plurality of bacteria and refining the machine learning model.

7. The method of claim 1, wherein the machine learning model is trained in an unsupervised process.

8. The method of claim 1, wherein the machine learning model is a deep learning based model.

9. A computational method for generating a therapeutic composition machine learning model, wherein the method comprises:
(a) compiling data from a plurality of bacterial strains in a computer database system, wherein the data comprises
(1) genomic sequence data of a plurality of bacterial strains; and
(2) experimentally derived therapeutic composition-host sensitivity profiles of the bacterial strains experimentally derived from a plurality of therapeutic compositions;
(b) training a machine learning model using the genomic sequence data of a plurality of bacterial strains and the experimentally derived therapeutic composition-host sensitivity profiles on a CPU and a memory unit of a computer system;
(c) storing a therapeutic composition machine learning model configured to receive a query bacterial genome and select at least therapeutic composition estimated to be sensitive to the bacterial genome based on the trained machine learning model.

10. The method as claimed in claim 9, wherein the at least therapeutic composition comprises at least one phage, at least on antibiotic, at least one bactericide or a combination.

11. The method of claim 9, wherein the machine learning model is iteratively trained using a supervised learning or reinforcement learning method.

12. The method of claim 9, wherein the machine learning model is a deep learning model.

13. The method of claim 9 further comprising receiving genomic sequence data of a plurality of phage strains; and the machine learning model is trained using the received genomic sequence data of a plurality of phage strains.

14. The method of claim 9, further comprising generating therapeutic composition-host sensitivity sequences by:
(1) identifying common genomic sequence patterns shared between the bacterial strains having similar or identical therapeutic composition-host sensitivity profiles; and/or
(2) identifying dissimilar genomic sequence patterns shared between the bacterial strains having dissimilar therapeutic composition-host sensitivity profiles; and
characterizing each bacterial strain by associating the therapeutic composition-host sensitivity sequences with therapeutic composition-host sensitivity profiles and generating a prediction profile for therapeutic composition-host specificity for each bacterial strain.

15. The method of claim 1, wherein the machine-learning model incorporates Neural network analysis, including deep Neural Network learning or Artificial Neural network analysis, or classic models, such as, Bayesian, Gaussian analysis, regression analysis, and/or Tree analysis.

16. The method of claim 5, wherein the experimentally derived therapeutic composition-host sensitivity data is generated by performing a plaque assay.

17. The method of claim 16, wherein the size, cloudiness, clarity and/or presence of a halo of a plaque is measured.

18. The method of claim 5, wherein the experimentally derived therapeutic composition-host sensitivity data is generated using a photometric assay selected from the group consisting of fluorescence, absorption, and transmission assays.

19. The method of claim 1, further updating the machine learning model comprising receiving:
(1) additional genomic sequence data of a plurality of bacterial strains; and
(2) experimentally derived therapeutic composition-host sensitivity profiles of the additional bacterial strains experimentally derived from a plurality of therapeutic compositions; and
retraining the machine learning model.

20. A computer implemented method for predicting therapeutic composition-host sensitivity of a query bacterium, the method comprising:
(a) receiving the machine learning model of claim 1;
(b) receiving genomic sequence data of the query bacterium;
(c) predicting a Therapeutic composition-host sensitivity of the query bacterium based on the machine learning model.

21. A method for selecting a therapeutic composition, wherein the method comprises selecting at least one therapeutic composition based on a profile match score generated from a query bacterial genome provided as input to the machine learning model of claim 1, wherein a higher profile match score represents a higher therapeutic composition sensitivity.

22. The method of claim 20, wherein multiple therapeutic compositions are selected.

23. The method of claim 22, wherein the multiple therapeutic compositions are formulated in a pharmaceutically acceptable composition.

24. The method of claim 19, wherein the selected therapeutic composition has a different host range.

25. The method of claim 19, wherein the selected therapeutic composition comprise a mixture of therapeutic compositions having broad host range and therapeutic compositions having a narrow host range.

26. The method of claim 19, wherein the selected therapeutic compositions act synergistically with one another.

27. The method of claim 19, wherein the therapeutic compositions have an activity selected from:
(a) delay in bacterial growth;
(b) lack of appearance of phage-resistant bacterial growth;
(c) less virulent;
(d) regain sensitivity to one or more drugs; and/or
(e) display reduced fitness for growth in the subject.

28. A composition comprising the therapeutic composition of claim 19.

29. A method of treating a bacterial infection in a subject in need thereof or a bacterial contamination comprising administering to the subject an effective amount of the composition of claim 28.

30. The method of claim 29 wherein the bacterial infection to be treated or bacterial infection is selected from the group consisting of wound infections, post-surgical infections, and systemic bacteremias.

31. The method of claim 29, wherein the bacterial infection and/or contamination is caused by a bacteria selected from “ESKAPE” pathogens (Enterococcus faecium, Staphylococcus aureus, Klebsiella pneumonia, Acinetobacter baumannii, Pseudomonas aeruginosa, and Enterobacter sp).

32. A system comprising: one or more processors; memory; and one or more programs, wherein the one or more programs are stored in the memory and configured to be executed by the one or more processors, the one or more programs including instructions for carrying out claim 1.

33. The method of claim 1, wherein at least one of the bacterial strains of the plurality of bacterial strains, the query bacterial genome, and/or the bacterial infection is (are):
a) multidrug resistant;
b) a clinical bacterial isolate causing infection in a subject;
c) a clinical bacterial isolate causing infection in a subject and is multidrug resistant;
d) obtained from bona-fide human infections; or
e) obtained from a diverse source.

34. The method of claim 33, wherein the diverse source is selected from the group consisting of soil, water treatment plants, raw sewage, sea water, lakes, rivers, streams, standing cesspools, animal and human intestines, and fecal matter.

35. A machine learning model created according to the method of claim 1.

36. Use of the machine learning model of claim 35 to predict therapeutic composition-host sensitivity to a query bacteria.
